(19) United States
(12) Patent Application Publication
(10) Pub. No.: US 2012/0232016 A1
(43) Pub. Date: Sep. 13, 2012

(54) METHOD AND SYSTEM TO DETECT AND DIAGNOSE ALZHEIMER'S DISEASE

(76) Inventor: PAUL D. COLEMAN, New River, AZ (US)

(21) Appl. No.: 13/043,182

(22) Filed: Mar. 8, 2011

(51) Int. Cl. A61K 38/00; G06F 17/18; G01N 33/48; A61P 25/28; G06F 19/00

(52) U.S. Cl. 514/17.8; 702/19

(57) ABSTRACT

Various embodiments provide methods for the detection, the diagnosis, and/or the prediction of disease onset of Alzheimer's disease. Methods for determining a state of Alzheimer's disease are provided. Accordingly, these methods can comprise the steps of placing a sample comprising at least one blood component onto a substrate, labeling the sample to identify at least one epigenetic marker and at least one of an inflammation marker and a cellular stress marker; determining an amount of the markers; performing a multivariate statistical analysis to produce an output value; comparing the output value to a reference value; and determining a state of Alzheimer's disease.

### STATEMENT REGARDING FEDERAL RIGHTS

This invention was made in part with government support under grants from the National Institute of Aging (Contract nos: AG09016, RO1 AG14411) and Alzheimer Disease Center (Contract no. AG08665). The U.S. government may have rights in this invention.

### BACKGROUND

Dementia and senility were once accepted as part of the natural aging process. In 1906, Dr. Alois Alzheimer reported histopathologic changes that he had found during the post-mortem examination of a patient suffering from senile dementia. Those changes are recognized today as the neurofibrillary tangles and amyloid plaques that are the hallmarks of Alzheimer's disease. Alzheimer's disease is characterized by progressive neurodegeneration ultimately resulting in dementia and death.

Today, while the ultimate pathology of Alzheimer's disease is fairly well established, effective diagnostic methods and treatment modalities remain elusive because of the complex biological basis for the etiology and pathogenesis of the disease. Scientists and clinicians lack reliable diagnostic tests due to the absence of biologically specific screening techniques. Clinical diagnostic techniques for Alzheimer's disease currently rely on screening individuals displaying symptoms of dementia by excluding other possible causes such as depression, poor nutrition, other dementing conditions (e.g., Parkinson's disease with dementia), or drug interactions. These qualitative and unspecific methods often leave Alzheimer's disease misdiagnosed or unrecognized until later stages in the disease when treatments may be less effective. Early detection and treatment of Alzheimer's disease continues to be the best hope for successful treatment that may delay symptoms and extend a patient's quality of life.

### SUMMARY

Various embodiments provide methods for the detection, the diagnosis, and/or the prediction of disease onset of Alzheimer's disease. Methods for determining a state of Alzheimer's disease are provided. Accordingly, these methods can comprise the steps of placing a sample comprising at least one blood component onto a substrate labeling the sample to identify at least one epigenetic marker and at least one of an inflammation marker and a cell stress marker; determining an amount of the markers; performing a multivariate statistical analysis to produce an output value; comparing the output value to a reference value; and determining a state of Alzheimer's disease.

Further areas of applicability will become apparent from the description provided herein. It should be understood that the description and specific examples are intended for purposes of illustration only and are not intended to limit the scope of the present teachings.

### BRIEF DESCRIPTION OF THE DRAWINGS FIGURES

The drawing figures described herein are for illustration purposes only and are not intended to limit the scope of the present teachings in any way. The present teachings will become more fully understood from the detailed description and the accompanying drawing figures wherein:

FIG. 1 is a bar graph illustrating univariate analysis of clinical data relating to the quantification of changes in the levels of inflammation markers, in accordance with one embodiment;

FIG. 2 is a bar graph illustrating multivariate analysis of clinical data relating to the quantification of changes in the levels of cellular stress markers, in accordance with one embodiment;

FIG. 3 is a bar graph illustrating univariate analysis of clinical data relating to the quantification of changes in the levels of epigenetic markers, in accordance with one embodiment;

FIG. 4 is a distribution plot illustrating clusters resulting from multivariate analysis of clinical data relating to the quantification of changes in the levels of inflammation markers, in accordance to one embodiment;

FIG. 5 is a distribution plot illustrating clusters resulting from multivariate analysis of clinical data relating to the quantification of changes in the levels of stress, in accordance to one embodiment;

FIG. 6 is a distribution plot illustrating clusters resulting from multivariate analysis of clinical data relating to the quantification of changes in the levels of epigenetic markers, in accordance with one embodiment; and

FIG. 7 is a table illustrating the percent of patients correctly classified with Alzheimer's disease risk based on the multivariate analysis, in accordance with one embodiment.

### DESCRIPTION

The following description is merely exemplary in nature and is not intended to limit the present teachings, applications, or uses. The description of specific examples indicated in various embodiments of this disclosure are intended for purposes of illustration only and are not intended to limit the scope of the teachings disclosed herein. Moreover, recitation of multiple embodiments having stated features is not intended to exclude any other embodiments having additional features or any other embodiments incorporating different combinations of the stated features. As used herein, the phrase “at least one of A, B, and C” should be construed to mean a logical (A or B or C), using a non-exclusive logical or. As used herein, the phrase “A, B and/or C” should be construed to mean (A, B, and C) or alternatively (A or B or C), using a non-exclusive logical or. It should be understood that steps within a method may be executed in different order without altering the principles of this disclosure.

The drawings described herein are for illustrative purposes only of selected embodiments and not all possible implementations, and are not intended to limit the scope of any of the various embodiments. It is understood that the drawings are not drawn to scale. For purposes of clarity, the same reference numbers will be used in the drawings to identify similar elements.

Various embodiments of the invention provide methods, apparatus, systems, and kits for detecting, diagnosing, predicting disease onset, and/or monitoring the progression of Alzheimer's disease (hereinafter “AD”). A detailed description of various embodiments, namely a method and system for detecting, diagnosing, predicting disease onset, and monitoring the progression of AD, is provided as a specific enabling disclosure that may be generalized to any application of the disclosed methods and systems in accordance with various embodiments described herein. Furthermore, the detailed description of various embodiments includes the best mode known to the inventors at the time of filing this application.

Various embodiments provide detecting, diagnosing, predicting disease onset, and monitoring the progression of AD through gene expression and gene product profile changes in blood components, such as leukocytes. Leukocytes may comprise any leukocyte subtype such as lymphocytes, neutrophils, basophils, and macrophages. In one embodiment, a method may comprise detecting gene expression profile changes in leukocytes, such as, for example, genes regulating inflammation, cellular stress, and epigenetic mechanisms.

In accordance with various embodiments, the levels of gene transcripts or gene products may be altered in the leukocytes of patients with AD. Further, the levels of gene transcripts or gene products that indicate AD may vary within patient groups due to genetic heterogeneity. In one embodiment, changes in the levels of gene transcripts or gene products for genes related to epigenetic mechanisms and at least one of inflammation response and cellular stress response may be detected in the leukocytes of patients in the early stages of AD, where the disease has not yet manifested to the degree that it may be diagnosed using the conventional methods of diagnosis.

AD may have a prolonged prodromal, preclinical phase in which brain damage on the molecular scale may be occurring for decades before clinical presentation of disease symptoms appear. Studies based on linguistics and visual memory of AD patients with documentation such as an autobiography or medical records from their youth suggest that neuropathology may begin in some patients as early as their 20s. Such data indicates that a long phase may exist in which symptoms are subtle and not yet clinically overt. In one embodiment, the detection of changes in the expression of key genes that are affected by AD can provide an accurate diagnosis of preclinical AD needed for beginning a course of treatment to ultimately prevent the emergence of symptoms that decrease quality of life.

The differential diagnosis of neurologic disorders such as AD may comprise performing a variety of conventional methods of diagnosis for elucidating the cause of mental impairment when symptoms become apparent. For example, conventional methods of diagnosis may comprise the performance of various qualitative tests by a clinician such as an evaluation of a patient's problem solving skills, attention span, counting skills, and memory to determine whether damage has occurred to specific areas of the brain. Further, a clinician may systematically rule out causes of the mental impairment by investigating a patient's medical history, such as for indications of previous trauma, family history of neurological disorders, medications, and psychosocial history, such as marital status, living conditions, employment, sexual history, and important life events that may indicate psychological causes such as depression. Through a process of elimination for alternative causes of the mental impairment or dementia, a clinician may begin to suspect AD.

However, a clinical diagnosis of AD may not be accurate and as a result, a patient may be on a treatment plan for AD, which may not treat the root cause of the symptoms, since the patient really has a non-AD disease or nutrient deficiency. For example, a patient of a senior age and with low vitamin B levels may exhibit symptoms of AD but not have AD. A treatment plan for AD most likely will not help this patient. However, treatments of vitamin B12 may help the patient have a better quality of life and may diminish the AD-like symptoms. If this patient is incorrectly clinically diagnosed with AD, the AD treatment plan will not affect the root cause of the patient's symptoms. An incorrect diagnosis of AD for a patient exhibiting at least some symptoms may be as high as 50% in some parts of the US. Even at the federally funded Alzheimer's Disease Centers, which reside at about 29 major medical institutions across the US, the incorrect diagnosis of AD for a patient exhibiting at least some symptoms may be 10% to 15%.

AD cannot be definitively diagnosed until brain tissue is examined after death for the presence of neurofibrillary tangles and amyloid plaques. While the examination of a living patient's brain tissue is not generally feasible or ethical, some microscopic changes to the brain in the later stages of AD may be detected using other conventional methods of diagnosis such as Computed tomography (CT) scanning, Nuclear Magnetic Resonance Imaging (MRI), and Positron Emission Tomography (PET). CT, MRI, and PET techniques may show changes in the brain that are characteristic of late stage AD such as atrophy of the brain, changes in brain activity, and blood vessel structure. Consequently, such techniques cannot detect the earliest stages of the disease where changes remain on a biochemical level inside the neuronal cells of brain tissue.

Expression changes in thousands of genes, spanning multiple biologic pathways, have been reported in pathologically-vulnerable regions of the AD brain. For example, changes to the molecular pathways for energy metabolism, inflammation, and cell cycle regulation have been reported that are believed to contribute to the pathogenesis of AD. These changes in gene expression are widespread in AD, but lack the elucidation of a common over-arching principle explaining the modification of gene expression across many different seemingly unrelated molecular pathways. Further, genetic heterogeneity within patient populations may complicate the detection of AD by masking the predictive value of individual genes.

While genetic changes to brain tissue affected with AD become elucidated, a non-invasive diagnostic test for AD remains elusive. The development of a diagnostic method for Alzheimer's disease based on the decreased incidence of markers in brain tissue is impractical for a variety of reasons such as the invasiveness and procedural risk of obtaining brain tissue and its associated high cost. Detecting the disease in its earliest stages makes such an approach highly problematic as the patient may be exhibiting only vague symptoms of the disease or no symptoms at all, which may make the procedural risk and cost of diagnosis unjustifiable.

The development of a noninvasive diagnostic technique for evaluating genetic profile changes characteristic of AD is thus problematic and infeasible in brain tissue. Basing the diagnostic technique on easily obtainable biological samples that may be collected at routine doctor's visits, such as blood, urine, a mouth swab, or a hair sample presents advantages in terms of convenience, cost control, and reduced procedural risk. However, according to the literature and other medical sources, no such diagnostic techniques currently exist.

According to various embodiments, general biological functions of the cell have been identified, which are affected by AD in cells outside the nervous system, such as, for example, peripheral blood cells. In one embodiment, the inflammatory responses and the associated pathway molecules may be altered in a variety of leukocyte classes in AD patients. In one embodiment, cell stress including, but not limited to, oxidative stress pathways, may occur in leukocyte cells of AD patients.

As disclosed herein, predicting or diagnosing an AD disease state by evaluating particular relevant individual markers shown to be modulated in the leukocytes of AD patients through univariate studies may reveal differences between AD samples and non AD control samples, but may significantly overlap between samples and diminish the utility of assaying single molecules as markers. However and in accordance to various embodiments, multivariate statistical analysis of multiples of the same relevant markers can effectively and clearly distinguish AD from control samples. Thus, in one embodiment, multivariate statistical analysis of particular relevant markers modulated by AD can in effect rescue the significance of these markers for making an accurate differential diagnosis of AD.

The inflammation, cellular stress, and epigenetic mechanisms discussed herein can be considered in terms of biomarkers. As known to those skilled in the art, the term “marker” is generally accepted as any specific organic biomolecule that is differentially present in a patient having one phenotypic status (ie., a disease state) as compared to a patient having another phenotypic status (ie., not having the disease state). The marker can be considered to be differentially present when comparing patients with different phenotypic statuses if the mean or median level of the marker is calculated to be statistically significant.

The marker can be detected by a biochemical test, or an analytical test, or a combination thereof. The marker can be, for example, a gene transcript such as mRNA, a gene sequence, a modification to a gene such as for example methylated DNA, or a gene product such as a protein or a peptide. A marker may indicate a presence or absence of an enzyme in a sample, and in some cases the marker may be used to determine a concentration of the enzyme in the sample. Also, a marker may indicate, for example, an activity of a biochemical reaction in a sample. Still further, a marker may indicate, for example, a presence or absence of protein in a sample, and in some cases the marker may be used to determine a concentration of protein in the sample.

Markers can exist in a sample in a plurality of different forms. The forms of the marker may result from cellular processes such as pre- and post translational modifications. Pre-translational modifications include, but are not limited to, allelic variants, spliced variants, and edited RNA forms of transcripts. Post translational modifications of gene products include, but are not limited to, proteolysis, glycosylation, phosphorylation, oxidation, and acetylation. Protein aggregation, complexation with other proteins, polymerization, denaturation, and other processes are contemplated. In various embodiments, any form of the marker may be detected.

Neuroinflammation of brain cells such as microglia and astrocytes is associated with the AD pathology of brain tissue. The activation of local and peripheral inflammatory responses is related to the degeneration of brain tissue and the deposition of highly insoluble amyloid beta peptides in an AD affected brain. Further, inflammation related proteins have been found to co-localize with the neurofibrillary tangles and amyloid-β plaques that characterize AD. The role of inflammation in AD pathogenesis has encouraged research into the use of anti-inflammatory therapeutics as a treatment to slow the progression of the disease.

Activation of the pro-inflammatory complement system has been implicated as a cause of neurodegeneration in traumatic brain injury and AD diseased brain tissue. The complement system comprises approximately thirty effector proteins that circulate in blood plasma until activated by antibody-antigen complexes. For example, a complement protein may remain inactive until it binds an antibody-antigen complex, causing a sequential enzymatic activation cascade, which results in cytotoxicity and death of the antigen. In accordance with one embodiment, amyloid-β plaques that result from AD can activate the complement system, triggering the recruitment of phagocytes, such as microglia, to the site of the plaques through activation of complement proteins, such as Complement 5 (C5a).

Cytokines are small proteins secreted by a variety of cell types that are involved in mediating immune responses. Cytokines are typically secreted by cells to modulate the behavior of other target cells by binding to cytokine receptors on the target cells. Some cytokines, referred to as chemokines, may stimulate the migration and activation of cells such as phagocytic cells and lymphocytes during immune modulation and/or inflammatory responses. In various embodiments, assays quantifying the amount of cytokine protein present in a sample may not be limited to assaying the protein isolated from leukocytes, but may also assay protein in a blood plasma fraction of a whole blood sample or assay total protein from a whole blood sample directly.

As used herein, the term “inflammation marker” is defined as at least one of a complement marker, a cytokine receptor marker, and a cytokine marker. Examples of a complement marker can include, but are not limited to, the gene and gene products of complement C5 (C5). Examples of cytokine receptor markers can include, but are not limited to, the gene and gene products of interleukin 10 receptor (IL10Ra) and interleukin 17 receptor (IL17Ra). Examples of cytokine markers can include, but are not limited to, the gene and gene products of interleukin 8 (IL8—a chemokine), leukemia inhibitory factor (LIF), and tumor necrosis factor-alpha (TNF∝).

Genes and gene products that are modulated in response to cellular stress may also function as statistically significant markers in the prediction and diagnosis of AD disease states. Cellular stress may include environmental factors such as nutritional deficiencies and temperature extremes. Cellular stress may also be inflicted by infections in which cells must synthesize large amounts of particular cell mediated immune response proteins to combat the infection. Additional sources of cellular stress may include oxidative damage and DNA damage.

As used herein, the term “cellular stress marker” is defined as at least one of an iron binding marker, a cellular metabolism marker, a chaperone marker, a cyclooxygenase marker, and a protease inhibitor marker. Examples of iron binding markers can include, but are not limited to, the gene and gene products of Heavy Chain Ferritin (H-Ferritin), Light Chain Ferritin (L-Ferritin), and transferring receptor (TfR). Examples of cellular metabolism markers can include, but are not limited to, the gene and gene products of glyceraldehyde-3-phosphate dehydrogenase (GAPDH). Examples of chaperone markers can include, but are not limited to, the gene and gene products of heat shock protein 27 (HSP27) and heat shock protein 90 (HSP90). Examples of cyclooxygenase markers can include, but are not limited to, the gene and gene products of cyclooxygenase-1/Prostaglandin-endoperoxide synthase 1 (COX1) and cyclooxygenase-2/Prostaglandin-endoperoxide synthase 2 (COX2). Examples of protease inhibitor markers can include, but are not limited to, the gene and gene products of alpha1-antichymotrypsin (∝-ACT, also identified as SERPINA3, a serine protease inhibitor).

Epigenetic mechanisms may account for, or contribute to, modulating global gene expression in a cell across different pathways. For example, epigenetic mechanisms causing changes to chromatin or DNA expression such as histone modification, binding of non-histone proteins, or DNA methylation, may be capable of causing global changes to gene expression that may be specific to AD. Epigenetic mechanisms may orchestrate widespread changes in cell phenotype by modifying the transcription of genes involved in many biological pathways across a genome.

Epigenetic mechanisms may involve changes in the micro- and macro-structure of chromatin, a complex of DNA, chromosome proteins, and histone proteins in which the histone proteins are tethered together in structures around which double-stranded DNA is wound. Conformational changes in histone proteins or modifications of the way in which DNA wraps around the histones may then differentially alter access of the transcriptional machinery to some genes while leaving access to other genes intact.

Although there are multiple mechanisms by which histones are modified, including methylation, phosphorylation, ubiquitination, sumoylation, citrullination, ADP-ribosylation, and other post-translational modifications of the amino acids that make up histone proteins, histone acetylation is one of the most ubiquitous and well studied. Histone acetyltransferases (HATs) catalyze the transfer of an acetyl group from acetyl-coenzyme A to lysine residues on the N-termini of histone proteins. As a result of acetylation, the positive charge of the histone proteins is neutralized, decreasing interactions of the histone protein tails with negatively-charged phosphate groups of associated DNA. This conformational relaxation of the chromatin permits access to and transcription of genes within the complex. Conversely, the histone deacetylases (HDACs) transfer acetyl groups from acetylated histone proteins back to coenzyme A, producing a more condensed chromatin state and decreased or silenced gene transcription.

DNA methylation comprises one type of epigenetic mechanism that modifies DNA, resulting in changes in gene expression. Adjacent cytosine-guanine dinucleotides (CpGs) within DNA sequences may be methylated by proteins called DNA methyltransferases. Methylation of cytosine-guanine dinucleotide pairs (CpGs) may inhibit the access of the cell's transcriptional machinery to the promoter region of the gene containing the methylated CpG sequence. Methylation may occur within the coding region of a gene or in repetitive DNA sequences that may flank a gene. Such methylation may alter gene expression even if it occurs at some distance from the promoter region.

Highly methylated genes may exhibit a decrease or repression in gene expression. Conversely, mechanisms that demethylate CpGs leading to hypomethylated DNA, may lead to an upregulation of gene expression. However, these trends are not universal as exceptions have been found in which genes that are hypomethylated exhibit repressed gene expression and genes that are hypermethylated are upregulated. Consequently, the expression of a particular gene and resulting changes in protein levels must be assayed to verify the effect of methylation on that particular gene.

DNA methylation is highly interactive with histone acetylation and the other histone-modifying mechanisms. Adjacent CpGs within DNA can be methylated by the actions of the DNA methyltransferases, DNMT1, DNMT2, DNMT3a/b, and DNMT4. In mammals, DNMT1 appears to be primarily involved in maintenance methylation of hemimethylated DNA after DNA replication, whereas DNMT3a and DNMT3b are involved in de novo methylation. DNMT2 is typically considered to be an RNA methyltransferase, although it also has 5-cytosine DNA methyltransferase activity and forms denaturant-resistant complexes with DNA. The methyl group that is transferred to cytosine by the DNMTs ultimately derives from folate through its interactions with S-adenosylmethionine and, further upstream, the homocysteine-methionine cycle.

Through these processes, approximately 70% of CpG dinucleotides within the human genome are methylated. Although methylation can take place at any CpG site on a gene, it may be particularly important with respect to CpG-rich stretches (CpG islands) within the promoter region. Some 50,267 CpG islands exist in the human genome, with 28,890 in simple repeat and low complexity sequences that are masked.

A second, linked mechanism by which DNA methylation may modify gene expression is through methyl-cytosine-binding complexes (MeCPs) such as MeCP2. When bound to methylated DNA, MeCP2 has been shown to recruit HDACs, which, as noted earlier, may then induce a more condensed chromatin state and decreased or silenced gene transcription. Mutations of the MeCP2 gene cause Rett's Syndrome, with dysregulation of neural development, mental retardation, and motor dysfunction.

MeCP1, a macromolecule made up of some 10 different peptides, including DOC1, may also act as a mediator between methylation and histone acetylation, recognizing and binding to CpG dinucleotides, recruiting HDACs, and inducing transcriptional repression. Unlike MeCP2, however, MeCP1 does not bind directly to methylated DNA, but to a single methyl-CpG-binding domain protein, MBD2. In addition to inducing histone modifications, MBD2-bound MeCP1 helps maintain the methylation status of CpGs by recruiting DNMT1. DNMT1 is then able to recognize and repair CpGs that have lost methyl groups on one DNA strand but not the other.

DNA methylation was once studied in the context of maintaining DNA methylation during cell divisions. However, the role of DNA methylation has been elucidated in postmitotic cells, including neurons in the field of neuroepigenetics. Neuroepigenetic studies of DNA methylation illustrate its role in mediating neuronal and synaptic plasticity, such as long-lasting modifications to hypothalamic neurons causing physiologic, memory, and behavioral changes in mice resulting from stress in early life.

The brain tissue of a patient with Alzheimer's disease known to be vulnerable to damage by the disease, such as entorhinal cortex layer II neurons, exhibits marked decreases in immunoreactivity for markers of DNA methylation and DNA methylation maintenance factors. For example, labeling neurons with an antibody to 5-methylcytosine and 5-methylcytidine, which are markers for methylated DNA, reveals dramatic decreases in immunoreactivity in brain tissue samples from patients with AD compared to samples from patient without the disease.

As used herein, the term “epigenetic marker” is defined as at least one of a DNA methylation marker, a histone modification marker, and a deacetylase marker. Examples of DNA methylation markers can include, but are not limited to, DNMT1, DNMT2, DNMT3a/b, MBD2, MBD3, 5-methylcytosine, 5-methylcytidine, MeCP, RPL26, p66, MTA2, RbAp48, DOC1, and combinations thereof. Examples of histone modification markers can include, but are not limited to, HDAC1, HDAC2, HAT1, and HDAC6, and combinations thereof. An example of a deacetylase marker can include, but is not limited to, NAD-dependent deacetylase sirtuin-1 (hereinafter referred to as SIRT1).

In accordance with various embodiments, the multivariate analysis of epigenetic, inflammation, and cellular stress markers, as discussed herein, can distinguish AD from non-AD and other neurodegenerative diseases in peripheral blood leukocytes. In one embodiment, the different expression levels of individual genes, as analyzed in a univariate studies, may not be as effective at discriminating a disease state as an analysis based on weighted sums of many gene products.

In various embodiments, multivariate statistical analysis can be used to elucidate whether the changes in transcript levels of pre-selected genes distinguish early onset AD from non-diseased control groups and from other neurodegenerative diseases, such as, for example, Parkinson's disease. Univariate analysis, such as the t-test, ANOVA, and the N test, considers only one variable at a time. Univariate analysis of individual gene transcripts may not distinguish early stages of AD from control samples without significant overlap.

Multivariate statistical analysis considers several variables in each sample. In one embodiment, multivariate analysis can determine the gene transcripts that best distinguishes the early stages of AD from non-diseased control groups and from other neurodegenerative disease control samples and assigns weights to each transcript. Relying on canonical discriminant analysis, multivariate analysis is based on the analysis of a correlation matrix that represents all the pre-selected transcripts and all the patient cases.

In one embodiment, the analysis may use the canonical analysis program in the SAS/STAT® software package. The analysis program was instructed that there are two (or more) groups (ie. AD group, non-AD control group, and non-diseased control group) and the weights for the pre-selected transcripts were determined. The ability of the weighted transcripts to distinguish the groups was assessed. The weight determined for each transcript and the expression level of that transcript were combined for each transcript for each person in each group. The gene expression levels modified by weights for each transcript can be combined to calculate an overall “AD risk score” for each person.

As disclosed herein, unexpected and surprising results have been obtained. The inventors have developed methodology that may predict the disease state of AD before the onset of disease symptoms by analyzing peripheral blood leukocytes from a patient's blood sample. These surprising and unexpected results are related to the discovery that multivariate discriminant analysis of particular inflammation, cellular stress, and epigenetic marker levels in leukocytes can be related to a disease state of AD in a patient with high specificity, distinguishing AD from non-diseased control groups and from other neurodegenerative diseases.

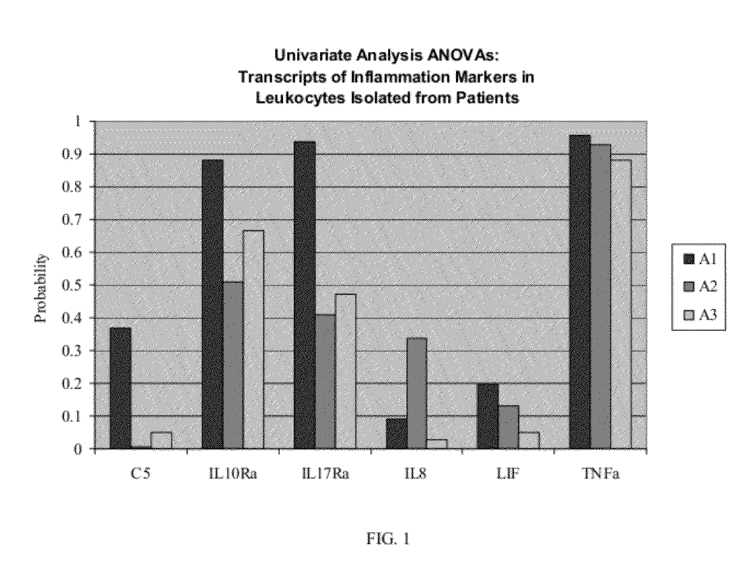

Now with reference to FIGS. 1-3, the ability of transcript levels for inflammation markers, cellular stress markers, and epigenetic markers to distinguish AD were assayed by micro array studies or qRTPCR analysis followed by univariate analysis of transcript levels, according to various embodiments. In accordance with various embodiments, blood samples can be obtained from three independent sets of samples from three different groups of people. Group A1 represents patients clinically diagnosed as at risk for developing AD (n=19) vs. those who actually converted to have clinically diagnosed AD. Group A2 represents patients clinically diagnosed as not at risk for developing AD (n=20) vs. those who actually converted to having clinically diagnosed AD. Group A3 represents patients who were clinically diagnosed as not at risk for developing AD (n=19) vs. those who were later clinically diagnosed as at risk for developing AD.

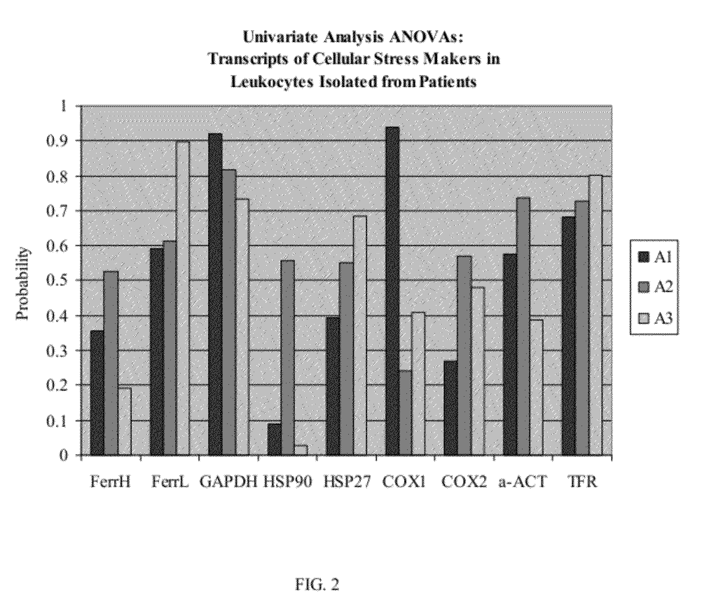

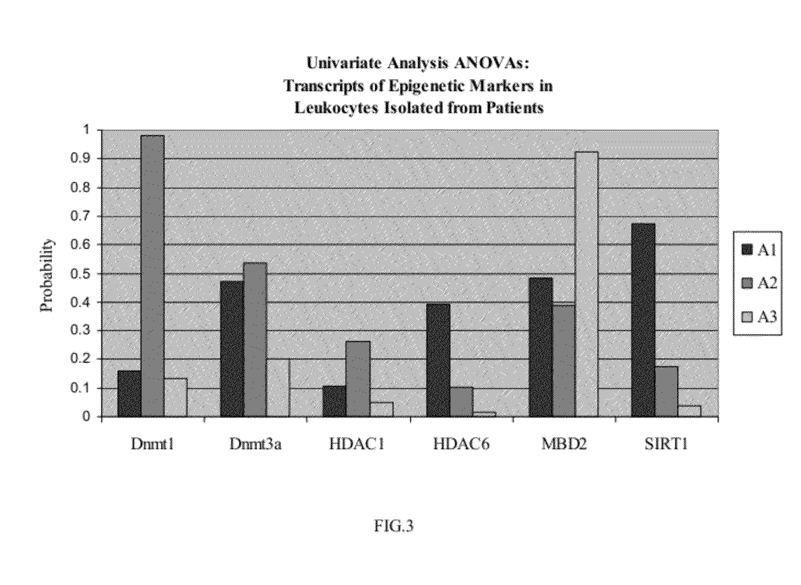

In various embodiments, leukocyte RNA from each sample can be hybrized to cDNA arrays containing cDNA clones comprising inflammation markers, cellular stress markers, and other cDNA clones with relevance to AD. cDNA arrays can be constructed with cDNA clones that may test the hypotheses that transcripts related to inflammation, cellular stress, and epigenetic mechanisms would be affected in leukocytes from AD cases. The dbEST database of the National Center for Biotechnology Information can be searched for relevant 3′ cDNA clones. In various embodiments, cDNA clones can be obtained from distributors of I.M.A.G.E. Consortium cDNA clones. As illustrated in FIGS. 1-3, 172 cDNA clones were represented in the arrays. The vertical axis designates the probability that each gene by itself distinguishes AD in each group of patients. The horizontal axis identifies the genes used in each analysis. FIG. 1 analyzed the genes Complement C5, IL10Ra, IL-17Ra, IL-8, LIF, and TNF-cc. FIG. 2 analyzed the genes FerrH, FerrL, GAPDH, HSP90, HSP27, COX1, COX2, cc-ACT, and TfR. FIG. 3 analyzed the genes Dnmt1, Dnmt2, HDAC1, HDAC6, MDB2, and SIRT1.

Univariate analysis by t-test revealed three transcripts to be statistically significantly different between AD and control samples. As illustrated in FIG. 2, these were: alpha-1 antichymotrypsin, and cycloogygenase II. Univariate analysis by N-test revealed one transcript to approach statistically significant difference between AD and control (p<0.08). This was the DNA repair enzyme, ERCC6 (data not shown). None of the transcripts revealed by either of these univariate statistics were sufficient in themselves to distinguish early AD from control without significant overlap (data not shown).

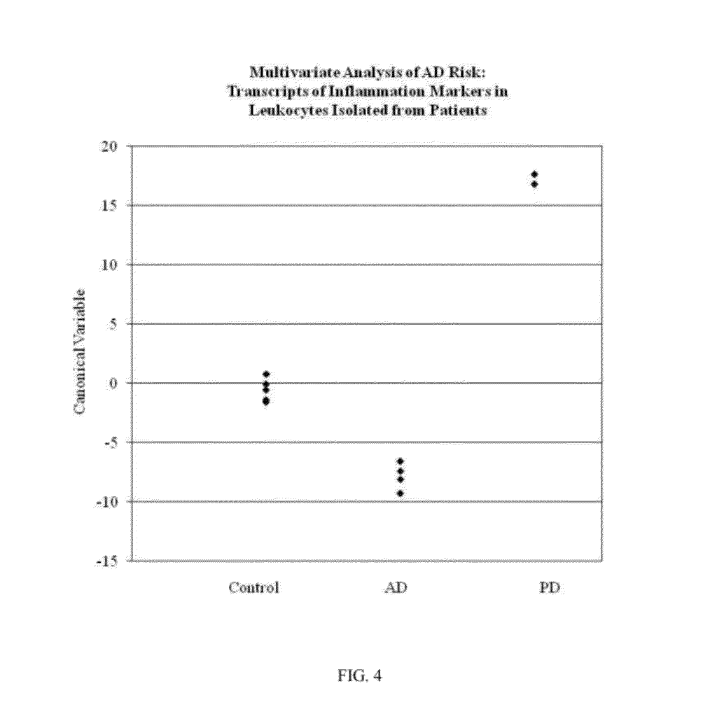

FIG. 4 illustrates the multivariate discriminant analysis of transcripts for gene products related to inflammation markers that can distinguish patients with AD from control non AD patients. Specifically, multivariate discriminant analysis of the gene transcripts for Complement C5, IL10Ra, IL17Ra, IL8, LIF, and TNF-cc were performed. The analysis reveals a clear distinction of AD samples from control samples, no overlap between the groups. The analysis also illustrates that Parkinson's disease is distinguished from non disease control patients and those with AD. Expression levels of transcripts were quantified by analysis using a micro-array and analyzed by multivariate discriminant analysis. Each diamond on the plot represents one case and the vertical axis is the canonical variable resulting from the analysis. From these groupings a pass/fail test for AD can be run for a patient's sample. In addition, from these groupings a pass/fail test for PD can be run for a patient's sample. In one embodiment, a threshold level can be set between the groups (such as, for example at about −5, as illustrated for the results in FIG. 4) and a result below the threshold predicts AD for the patient. In one embodiment, a threshold level can be set between the groups (such as, for example at about 10, as illustrated for the results in FIG. 4) and a result above the threshold predicts PD for the patient. These results may be combined with results from multivariate analysis of other gene sets. Furthermore, these results may be added to results from multivariate analysis of other gene sets for additional statistical analysis, such as, for example multivariate analysis, to render a risk score for a patient. Such a risk score can indicate or predict at least one of a risk not to contract AD, or a risk not to contract PD, or a risk not to contract either PD or AD, or a risk not to contract AD and PD. In addition, such a risk score can indicate or predict at least one of a risk to contract AD, or a risk to contract PD, or a risk to contract either PD or AD, or a risk to contract AD and PD.

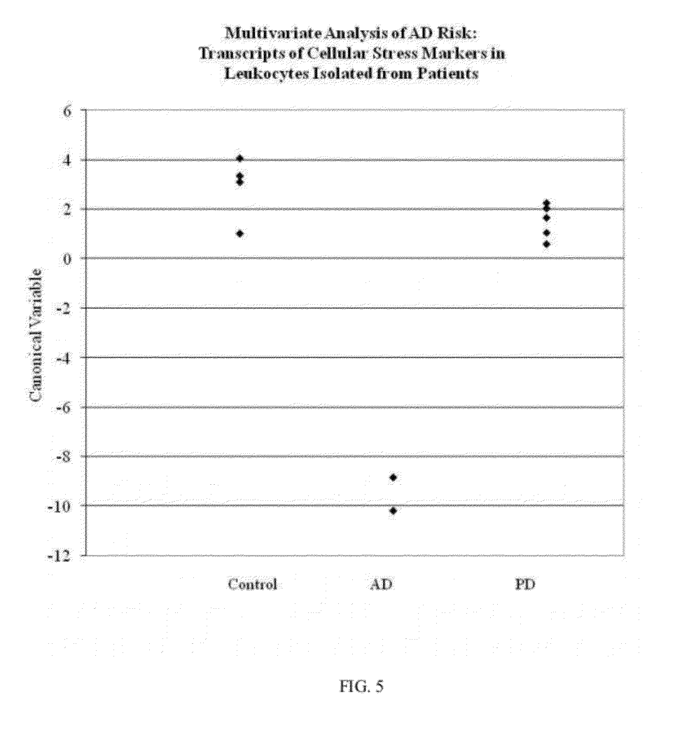

FIG. 5 illustrates the multivariate discriminant analysis of transcripts for gene products related to cellular stress markers that can distinguish patients with AD from control non AD patients. Specifically, multivariate discriminant analysis of the gene transcripts for FerrH, FerrL, GAPDH, HSP90, HSP27, COX1, COX2, ∝-ACT, and TfR were performed. The analysis reveals a clear distinction of AD samples from control samples, with no overlap between the groups. The analysis also illustrates that Parkinson's disease is not well distinguished from non disease control patients and those with AD for cellular stress markers. Expression levels of transcripts were quantified by analysis using a micro-array and analyzed by multivariate discriminant analysis. Each diamond on the plot represents one case and the vertical axis is the canonical variable resulting from the analysis. From these groupings a pass/fail test for AD can be run for a patient's sample. In addition, from these groupings a pass/fail test for PD can be run for a patient's sample. In one embodiment, a threshold level can be set between the groups (such as, for example at about −4, as illustrated for the results in FIG. 5) and a result below the threshold predicts AD for the patient. In one embodiment, a threshold level can be set between the groups (such as, for example at about 3, as illustrated for the results in FIG. 5) and a result above the threshold predicts PD for the patient. These results may be combined with results from multivariate analysis of other gene sets. Furthermore, these results may be added results from multivariate analysis of other gene sets for additional statistical analysis, such as, for example multivariate analysis, to render a risk score for a patient. Such a risk score can indicate or predict at least one of a risk not to contract AD, or a risk not to contract PD, or a risk not to contract either PD or AD, or a risk not to contract AD and PD. In addition, such a risk score can indicate or predict at least one of a risk to contract AD, or a risk to contract PD, or a risk to contract either PD or AD, or a risk to contract AD and PD.

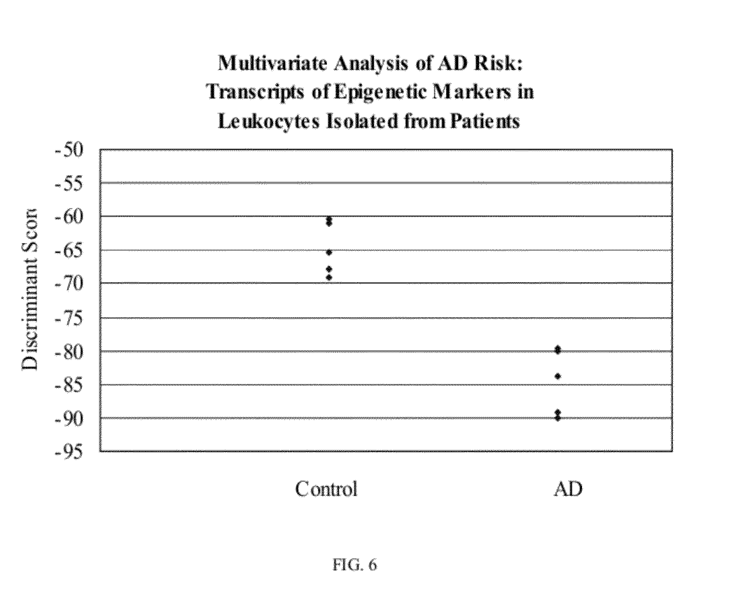

FIG. 6 illustrates the multivariate discriminant analysis of transcripts for gene products related to epigenetic markers that can distinguish patients with AD from control non AD patients. Specifically, multivariate discriminant analysis of the gene transcripts for DNMT1, DNMT3A, HDAC1, HDAC6, SIRT1, and MBD2 were performed. The analysis reveals a clear distinction of AD samples from control samples, with no overlap between the groups. Expression levels of transcripts were quantified by qRT-PCR and analyzed by multivariate discriminant analysis. Each diamond on the plot represents one case and the vertical axis is the risk score resulting from the analysis. From these groupings a pass/fail test for AD can be run for a patient's sample. In one embodiment, a threshold level can be set between the groups (such as, for example at about −75, as illustrated for the results in FIG. 6) and a result below the threshold predicts AD for the patient. These results may be combined with results from multivariate analysis of other gene sets. Furthermore, these results may be added results from multivariate analysis of other gene sets for additional statistical analysis, such as, for example multivariate analysis, to render a risk score for a patient. Such a risk score can indicate or predict at least one of a risk not to contract AD. In addition, such a risk score can indicate or predict at least one of a risk to contract AD.

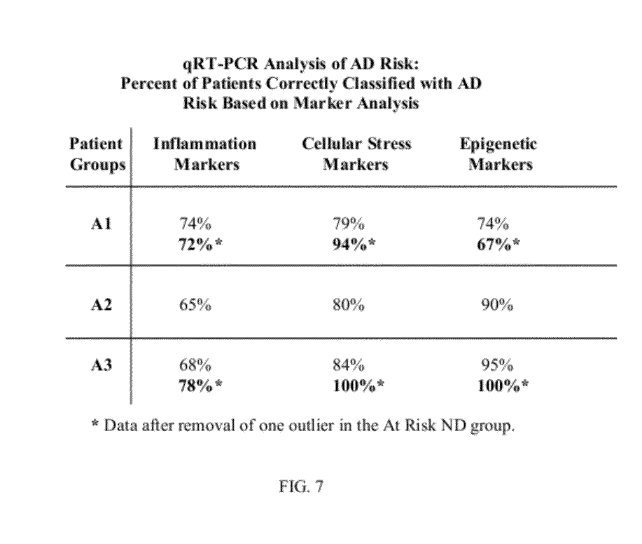

FIG. 7 is a table illustrating the ability of the inflammatory markers, cellular stress markers, and epigenetic markers to predict a subsequent diagnosis of AD in patients who were not showing symptoms AD, but later phenoconverted to AD. Patients in the study illustrated were a cohort of well characterized cognitively intact persons who were deemed to be at risk of developing AD by virtue of age 70 and above as well as at least one first degree relative having been diagnosed with AD. Over 1,000 longitudinal blood samples were collected to investigate the predictive value of applying the multivariate discriminant analytical approach to analyzing the inflammation markers, cellular stress markers, and epigenetic markers.

The percent of patients correctly classified as having AD by the multivariate discriminant analysis of transcript levels from qRT-PCR are shown. In accordance with various embodiments, blood samples can be obtained from three independent sets of samples from three different groups of people. Group A1 represents patients clinically diagnosed as at risk for developing AD vs. those who actually converted to have clinically diagnosed AD. Group A2 represents patients clinically diagnosed as not at risk for developing AD vs. those who actually converted to having clinically diagnosed AD. Group A3 represents patients who were clinically diagnosed as not at risk for developing AD vs. those who were later clinically diagnosed as at risk for developing AD. These groups are the same classifications as illustrated in FIGS. 1-6, however the population in each group is larger.

As illustrated in FIG. 7, the multivariate analysis of the transcripts from a patient's blood resulted in correctly classifying a patient with AD in group A1 72% of the time based on at least one inflammation biomarker, 94% of the time based one at least one cellular stress biomarker, and 67% of the time based on at least one epigenetic biomarker, with a removal of a patient with a blood iron disorder. The multivariate analysis of the results from analysis a patient's blood resulted in correctly classifying a patient with AD in group A1 74% of the time based on at least one inflammation biomarker, 79% of the time based one at least one cellular stress biomarker, and 74% of the time based on at least one epigenetic biomarker, including the patient with the blood iron disorder. It is not known if the blood iron disorder, some other unique disorder, poor sampling techniques, or errors in data analysis lead to this patient being an outliner. Further study is needed to determine how this patient became an outliner, thus this data point has been excluded from the multivariable analysis. A multivariate analysis of the results based on the at least one inflammation biomarker and the at least one epigenetic biomarker will increase the percentage of correctly classified patients with AD in group A1 to over 94%. A multivariate analysis of the results based on the at least one cellular stress biomarker and the at least one epigenetic biomarker will increase the percentage of correctly classified patients with AD in group A1 to over 94%. A multivariate analysis of the results based on the at least one inflammation biomarker and the at least one cellular stress biomarker and the at least one epigenetic biomarker will increase the percentage correctly classified patients with AD in group A1 to over 94%. In one embodiment, results from analyzing a patient's blood resulted in correctly classifying a patient with AD in group A1 is about 94% or greater. In various embodiments, results from analyzing a patient's blood correctly classified a patient with AD in group A1 is at least 99%, or is at least 98%, or is at least 97%, or is at least 95%, or is at least 90%, or is at least 87%, or is at least 84%, or at least 80%.

Also, as illustrated in FIG. 7, the multivariate analysis of the transcripts from a patient's blood resulted in correctly classifying a patient with AD in group A2 65% of the time based on at least one inflammation biomarker, 80% of the time based one at least one cellular stress biomarker, and 90% of the time based on at least one epigenetic biomarker. A multivariate analysis of the results based on the at least one inflammation biomarker and the at least one epigenetic biomarker will increase the percentage of correctly classified patients with AD in group A2 to over 90%. A multivariate analysis of the results based on the at least one cellular stress biomarker and the at least one epigenetic biomarker will increase the percentage of correctly classified patients with AD in group A to over 90%. A multivariate analysis of the results based on the at least one inflammation biomarker and the at least one cellular stress biomarker and the at least one epigenetic biomarker will increase the percentage of correctly classified patients with AD in group A2 to over 90%. In one embodiment, results from analysis a patient's blood resulted in correctly classifying a patient with AD in group A2 is about 90% or greater. In various embodiments, results from analyzing a patient's blood correctly classified a patient with AD in group A2 is at least 99%, or is at least 98%, or is at least 97%, or is at least 95%, or is at least 90%, or is at least 87%, or is at least 84%, or at least 80%.

Further as illustrated in FIG. 7, the multivariate analysis of the results from analyzing a patient's blood resulted in correctly classifying a patient with AD in group A3 78% of the time based on at least one inflammation biomarker, 100% of the time based one at least one cellular stress biomarker, and 100% of the time based on at least one epigenetic biomarker, with a removal of a patient with a blood iron disorder. The multivariate analysis of the results from analyzing a patient's blood resulted in correctly classifying a patient with AD in group A1 68% of the time based on at least one inflammation biomarker, 84% of the time based one at least one cellular stress biomarker, and 95% of the time based on at least one epigenetic biomarker, including the patient with a blood iron disorder. As discussed herein, it is not known if the blood iron disorder, some other unique disorder, poor sampling techniques, or errors in data analysis lead to this patient being an outliner. Further study is needed to determine how this patient became an outliner, thus this data point has been excluded from the multivariable analysis. A multivariate analysis of the results based on the at least one inflammation biomarker and the at least one epigenetic biomarker will increase the percentage of correctly classified patients with AD in group A3 to about 100%. A multivariate analysis of the results based on the at least one cellular stress biomarker and the at least one epigenetic biomarker will increase the percentage of correctly classified patients with AD in group A3 to about 100%. A multivariate analysis of the results based on the at least one inflammation biomarker and the at least one cellular stress biomarker and the at least one epigenetic biomarker will increase the percentage of resulted correctly classified patients with AD in group A3 to about 100%. In one embodiment, results from analyzing a patient's blood correctly classified a patient with AD in group A3 is about 100%. In various embodiments, results from analyzing a patient's blood correctly classified a patient with AD in group A3 is at least 99%, or is at least 98%, or is at least 97%, or is at least 95%, or is at least 90%, or is at least 87%, or is at least 84%, or at least 80%.

These data indicate a high level of accuracy in the ability of certain peripheral blood markers to predict Alzheimer's disease. In one embodiment, a multivariate analysis for all sets of biomarkers across all 3 groups may correctly classify that a patient has developed or will develop AD to about 100% accuracy, or is at least 99%, or is at least 98%, or is at least 97%, or is at least 95%, or is at least 90%, or is at least 87%. In one embodiment, a multivariate analysis for at least one epigenetic biomarker and at least one of a inflammation biomarker and an a cellular stress biomarker across all 3 groups may correctly classify that a patient has developed or will develop AD is about 100%, or is at least 99%, or is at least 98%, or is at least 97%, or is at least 95%, or is at least 90%, or is at least 87%. As illustrated, the multivariate discriminant analysis of inflammatory markers, cellular stress markers, and epigenetic markers distinguished at a high level between patients that are not at risk for AD from patients that are at risk converters or non-converter patients. These data also indicate that AD can be predicted across all groups: A1, A2, and A3. These data illustrate that the use of multivariate analysis on results related to at least one epigenetic biomarker, at least one cellular stress biomarker, and at least one inflammation biomarker from a patient's blood can predict later development of AD in a patient, who is either at risk or not at risk. In addition, these data illustrate that the use of multivariate analysis on results related to at least one epigenetic biomarker and at least one inflammation biomarker from a patient's blood can predict later development of AD in a patient, who is either at risk or not at risk. Furthermore, these data illustrate that the use of multivariate analysis on results related to at least one epigenetic biomarker and at least one cellular stress biomarker from a patient's blood can predict later development of AD in a patient, who is either at risk or not at risk. The percentages shown in bold represent data after removal of one outlier patient in the at risk group who was shown to have an iron metabolism disorder.

In accordance with various embodiments and with reference to the Figures discussed above, multivariate analysis of the transcripts related to inflammatory markers, cellular stress markers, and epigenetic markers can be used to distinguish Alzheimer's disease in peripheral blood leukocytes. These general classes of transcripts are modulated in brain tissue affected by AD brain and in peripheral blood leukocytes. In one embodiment, multivariate analysis of the transcript levels is shown to reproducibly predict AD where the univariate analysis of single transcripts cannot distinguish AD with statistical significance in blood cells. These data are consistent with the hypothesis that Alzheimer's disease has systemic components that affect selected classes of transcripts in easily obtainable peripheral cells. In one embodiment, transcripts within the inflammation, cellular stress, and epigenetic pathways other than those used here may also be useful in distinguishing AD and other classes of transcripts may also be useful.

Various embodiments provide methods for the detection, the diagnosis, the prediction of disease onset, and/or the monitoring the progression of AD by observing a present state of the levels of epigenetic markers and the levels at least one of inflammation markers and cellular stress markers in the leukocytes in a patient blood sample. According to various embodiments, the present state of epigenetic markers and the levels of the at least one of inflammation markers and cellular stress markers in leukocytes can be determined by either a direct measure of gene transcript levels of these markers or a measure of levels of their protein products.

In various embodiments, a method for determining the probability of developing Alzheimer's disease in a patient before the onset of symptoms, can comprise the steps of: obtaining a biological sample comprising at least one blood component from the patient; determining an expression level of at least one epigenetic marker and at least one of an inflammation marker and a cell stress marker; applying a multivariate statistical analysis to the expression levels of the at least one epigenetic marker and the at least one of the inflammation marker and the cell stress marker to produce an output value; comparing the output value to a reference value; wherein comparing the output value to the reference value results in a risk score; and determining the patient's risk of developing Alzheimer's disease based on the risk score.

In one embodiment, the at least one epigenetic marker is at least one of a DNA methylation marker, a histone modification marker, a methylated DNA binding protein marker, and a deacetylase marker. In one embodiment, the at least one inflammation marker is at least one of a complement marker, a cytokine receptor marker, and a cytokine marker. In one embodiment, the at least one cell stress marker is at least one of an iron-binding protein marker, a cellular metabolism marker, a protein chaperone marker, a cyclooxygenase marker, and a protease inhibitor marker. In one embodiment, the at least one epigenetic marker and the at least one of an inflammation marker and the cell stress marker are at least one epigenetic marker and at least one the inflammation marker and at least one cell stress marker. In one embodiment, the method can further comprise determining the patient's risk of developing Parkinson's Disease (PD) based on the risk score.

In various embodiments, a method for determining a state of Alzheimer's disease in a patient can comprise the steps of: obtaining a biological sample comprising at least one blood component from the patient; determining an amount of expression for at least one epigenetic marker and at least one of an inflammation marker and a cell stress marker in the biological sample; applying a multivariate statistical analysis to the amount of expression to produce an output value; comparing the output value to a reference value; and determining a state of Alzheimer's disease in the patient.

In one embodiment, the comparing the output value to the reference value results in a risk score representing the patient's relative risk of developing Alzheimer's disease. In one embodiment, the risk score distinguishes the state of Alzheimer's disease from a non-disease state in the patient from a state another neurological disease in the patient, such as but not limited to PD. In one embodiment, the method can further comprise determining a risk score for PD. In one embodiment, the method can further comprise determining a state of PD in the patient.

In various embodiments, the method can further comprise applying a label to the sample to identify at least one epigenetic marker and at least one of an inflammation marker and a cell stress marker. In one embodiment, the method can comprise placing the sample comprising the at least one blood component onto a substrate. In one embodiment, the determining an amount of the at least one epigenetic marker and the at least one of the inflammation marker and the cell stress marker comprises quantifying an intensity of the label.

In various embodiments, the at least one epigenetic marker and the at least one of an inflammation marker and the cell stress marker are at least one epigenetic marker and at least one the inflammation marker and at least one cell stress marker. In one embodiment, the method can further comprise preparing a treatment plan for the patient based on the state of AD. In one embodiment, the method can further comprise preparing a treatment plan for the patient based on the state of PD. In one embodiment, the method can comprise treating the patient with a therapeutic substance. In one embodiment, the at least one epigenetic marker is at least one of a DNA methylation marker, a histone modification marker, a methylated DNA binding protein marker, and a deacetylase marker. In one embodiment, the at least one inflammation marker is at least one of a complement marker, a cytokine receptor marker, and a cytokine marker. In one embodiment, the at least one cell stress marker is at least one of an iron-binding protein marker, a cellular metabolism marker, a protein chaperone marker, a cyclooxygenase marker, and a protease inhibitor marker.

In various embodiments, a method can further comprise the steps of: placing a second sample comprising the at least one blood component onto the substrate; determining a second amount of expression of the at least one epigenetic marker and the at least one of a inflammation marker and a cell stress marker; comparing the second amount expression of the at least one epigenetic marker and the at least one of the inflammation marker and the cell stress marker to at least one second reference value; applying a multivariate statistical analysis to the second amount of the at least one second reference value compared to the second amount of the at least one epigenetic marker and the at least one of the inflammation marker and the cell stress marker to produce a second output value; and determining a dosage of the therapeutic substance. In one embodiment, the method can further comprise evaluating the efficacy of the therapeutic substance. In one embodiment, the evaluating the efficacy of the therapeutic substance further comprises comparing the amount of the at least one epigenetic marker and the at least one of the inflammation marker and cell stress marker over a period of time. In one embodiment, the method can further comprise determining an updated state of Alzheimer's disease in the patient. In one embodiment, the method can further comprise developing a treatment plan based on the updated state of Alzheimer's disease in the patient.

In one embodiment, a method can comprise the steps of collecting a blood sample from a patient; isolating leukocytes or a portion thereof from the blood sample; isolating the total RNA from the leukocytes; reverse transcribing the RNA to produce cDNA; performing qRT-PCR using probes to amplify the transcripts for each marker, producing amplicons; quantifying the number of amplicons such as, for example, when the amplification process is in the exponential phase; normalizing the number of amplicons with an endogenous control such as beta glucuronidase (GUSB); and applying multivariate discriminant analysis to the number of amplicons to distinguish patients at risk for AD from patients not at risk for AD. In an exemplary embodiment, multivariate discriminant analysis may be used to distinguish patients at risk for AD from patients at risk for other neurological diseases such as, for example, PD.

In various embodiments of the methods, the qRT-PCR probes can comprise any oligonucleotide primer that can hybrize to a target transcript. In various embodiments, amplicons produced by the PCR reaction may be detected from the quantification of a fluorescent signal. For example, the PCR probes may depend on Förster Resonance Energy Transfer (FRET) to generate a fluorescent signal via the coupling of a fluorogenic dye molecule and a quencher moiety to transcript substrates. Exemplary probes include, but are not limited to, TaqMan® (Applied Biosystems, Foster City, Calif., USA), Molecular Beacons, Scorpions®, and SYBR® Green (Molecular Probes).

In various embodiments, methods can comprise the steps of collecting a blood sample; isolating leukocytes or a portion thereof from the blood sample; binding an antibody to at least one epigenetic marker and at least one inflammation and cellular stress marker located in the leukocytes; staining or otherwise labeling the antibodies bound to the markers; observing, or measuring, or quantifying an amount of stain or a signal from the labels bound to the antibodies; and performing a multivariate discriminant analysis on the amount of stain or the signal from the labels to distinguish patients at risk for AD from patients not at risk for AD or patients at risk for other neurological diseases such as, for example, PD.

In various embodiments of the methods, the stain or the label can comprise any moiety that can conjugate to an antibody that binds to the epigenetic marker, inflammation marker, or the cellular stress marker. In one embodiment, the epigenetic marker, inflammation marker, or the cellular stress marker may comprise a gene transcript such as mRNA, a gene sequence, a modification to a gene such as for example methylated DNA, or a gene product such as a protein or a peptide. In one embodiment, the epigenetic marker may comprise a methylated DNA site or to an epigenetic mechanism of DNA methylation, such as, for example but not limited to, methylation promoters, methylation inhibitors, methylation maintainers, and histone-related markers. Still further, in one embodiment, the stain or the label can comprise an antibody that binds to an antibody that binds to the epigenetic marker, inflammation marker, or the cellular stress marker.

The methods can include the addition of a label, such as a visible dye or fluorophore conjugated to a detecting secondary antibody for subsequent detected. For example, such a label may be detected by a human eye, with magnification, such as for example an optical microscope or without magnification. In another example, such a label may be detected by use of a reader, such as, for example but not limited to, a spectrometer, a flourometer, a fluorescence detector, a colorimeter, a densitometer, flow cytometer, an immunosorbent assay or other techniques that are familiar to those skilled in the art or are created in the future.

According to various embodiments, an immunoassay can be used to analyze a sample comprising a leukocyte or protein or DNA extract from a leukocyte and determination of an amount of at least one epigenetic marker and at least one of inflammation marker and cellular stress marker. Examples of such formats include an ELISA, radio-immunoassay, dot blot assay, slot blot assay, immunoprecipitation and protein quantification, immuno-PCR, and Western blot.

As described herein, relating to the epigenetic markers, DNA methylation is an epigenetic event that refers to the covalent addition of a methyl group, catalyzed by a family of DNMT enyzmes, to the 5-carbon of cytosine in a CpG dinucleotide. Methods for DNA methylation analysis can be divided roughly into two types: global and gene-specific DNA methylation analysis. According to various embodiments, for global DNA methylation analysis, methods which measure the overall level of methyl cytosines in the genome can include chromatographic methods and methyl accepting capacity assay. For gene-specific DNA methylation analysis, a large number of techniques have been developed. Most early studies used methylation sensitive restriction enzymes to digest DNA followed by Southern detection or PCR amplification. Recently, bisulfite reaction based methods have become very popular such as DNA methylation specific PCR (MSP), bisulfite genomic sequencing PCR. Additionally, in order to identify unknown DNA methylation hot-spots or methylated CpG islands in the genome, several genome-wide screening methods have been invented such as Restriction Landmark Genomic Scanning for Methylation (RLGS-M), and CpG island microarray.

Furthermore, a sample comprising a leukocyte can be analyzed by a variety of methods to determine an amount of at least one epigenetic marker and at least one of an inflammation marker and a cellular stress marker including but not limited to fluorescence detection, DNA sequencing gel, capillary electrophoresis on an automated DNA sequencing machine, microchannel electrophoresis, and other methods of sequencing, mass spectrometry, time of flight mass spectrometry, quadrupole mass spectrometry, magnetic sector mass spectrometry, electric sector mass spectrometry infrared spectrometry, ultraviolet spectrometry, palentiostatic amperometry or by DNA hybridization techniques including Southern Blots, Slot Blots, Dot Blots, and DNA microarrays, wherein DNA fragments would be useful as both “probes” and “targets,” ELISA, fluorimetry, Fluorescence Resonance Energy Transfer (FRET), SNP-IT, GeneChips, HuSNP, BeadArray, TaqMan assay, Invader assay, MassExtend, or MassCleave™ (hMC) method.

White blood cell (WBC) or leukocyte isolation from peripheral blood can be accomplished using a wide variety of methodologies, such as for example, but not limited to standard density gradient separation, commercially available evacuated separation tube systems, cell sorting systems, or other techniques familiar to those skilled in the art.

As can be appreciated by those skilled in the art, blood can be fractionated, and the different fractions of the blood can be used for different medical needs. Under the influence of gravity or centrifugal force, blood spontaneously sediments into three layers. At equilibrium, the top low-density layer is a straw-colored clear fluid called plasma. The bottom, high-density layer is a deep red viscous fluid comprising anuclear red blood cells (erythrocytes) specialized for oxygen transport. The intermediate layer is the smallest, appearing as a thin white band above the erythrocyte layer and below the plasma layer; this is called the buffy coat. The buffy coat itself has two major components, nucleated leukocytes (white blood cells) and anuclear smaller bodies called platelets (or thrombocytes).

Also, as can be appreciated by those skilled in the art, one way of obtaining white cells from whole blood is simply to allow EDTA-blood to settle in siliconized glasses and then pipette off the leukocyte-rich supernatant. Separating blood to isolate the WBC component or the leukocytes is well known to those skilled in the art. However in various embodiments, whole blood or a portion of blood that comprises leukocytes can be analyzed by methods described herein and without separating the WBC component or the leukocytes from the whole blood or the portion of blood that comprises leukocytes.

Various embodiments provide methods for determining a state of AD in a human. Accordingly, methods can comprise the steps of: placing a sample comprising at least one blood component onto a substrate; labeling the sample to identify at least one epigenetic marker and an inflammation and/or cellular stress marker; determining an amount of the at least one epigenetic marker and at least one of a inflammation marker and a cellular stress marker; applying a multivariate discriminant analysis to the amount of the at least one epigenetic marker and at least one of an inflammation marker and a cellular stress marker to produce an output value; and determining a state of AD. These methods can further comprise the step of separating blood into the at least one blood component and other blood components, to produce the sample comprising at least one blood component onto a substrate. In various embodiments, the at least one blood component comprises leukocytes. The sample can be from a patient.

Furthermore, these methods can comprise the step of preparing a treatment plan for a patient. In addition, these methods can comprise the step of treating the patient with a therapeutic substance. These methods can further comprise the steps of: placing a second sample comprising the at least one blood component onto the substrate; labeling the second sample to identify the at least one epigenetic marker and at least one of an inflammation marker and a cellular stress marker; determining a second amount of the at least one epigenetic marker and at least one of an inflammation marker and a cellular stress marker; applying a multivariate discriminant analysis to the amount of the at least one epigenetic marker and at least one of an inflammation marker and a cellular stress marker to produce an output value; and further determining a state of AD. An analysis of the second sample can be substantially simultaneous with the sample or the analysis can be later in time after the analysis of the sample. These methods can include the step of determining a dosage of a therapeutic substance to administer to the patient. These exemplary methods can comprise a step of observing a quantitative amount of the label. These methods can comprise binding an antibody to at least one epigenetic marker and an inflammation and/or cellular stress marker. Still further, these methods can comprise the step of introducing an antibody comprising a label to conjugate to the antibody.

In accordance to various embodiments, any of the methods discussed herein can be extended over time, such as for example, a longitudal study comparing a first set of a patient's results related to one or more epigenetic markers and at least one of an inflammation marker and a cellular stress marker at a first point in time to a second set of patient's results related to one or more epigenetic markers and at least one of an inflammation marker and a cellular stress marker at a second point in time. Such a comparison can provide one of a prediction or likelihood of developing AD. Such a comparison can provide a likely rate of developing AD. Still further, such a comparison can be useful in evaluating an efficacy of a therapeutic substance, as well as adjusting a dosage of such a therapeutic substance. Such a comparison can be part of a treatment plan. Although such results can be calculated by extrapolating from a single point measurement, at least two or more measurements taken some time apart as longitudinal data, would confirm the single point extrapolation or provide a new state of AD. For example, the measurement can be taken from one week to 2 years apart. However, the frequency of measurement could be about every 3 months, or about every 6 months, or about once a year, or about bi-annually. In various embodiments, a comparison can produce a difference (T0−T1) or a rate of change (T0−T1)/Time, where T0=marker amount or value at time zero, T1=amount at Time one, and Time=the amount of time between measurements.

Listed in Table 1 below are commercially available antibodies that may be useful in accordance to various embodiments. These commercially available antibodies may be useful in binding to an epigenetic marker, an inflammation marker, or a cellular stress marker in a leukocyte. These commercially available antibodies have specificity for individual epigenetic markers, inflammation markers, or cellular stress markers. However, a plurality of these commercially available antibodies or other similar antibodies not listed may be included in commercially available kits in accordance with various embodiments.

TABLE 1
Commercial Antibodies

| Antibody | Host/Type | Source/catalogue# | Antigen/Epitope |
|---|---|---|---|
| MBD2 | Goat polyclonal | Abcam/ab58241 | RNDPLNQNKGKPDLN |
| MBD3 | Mouse | Abcam/ab45027 | CKAFMVTDEDIRKQEE |
| | monoclonal | | |
| DOC1 | Rabbit polyclonal | Abcam/ab31794 | TSSQYRQLLSDYGPPS |
| DNMT1 | Rabbit polyclonal | Abcam/ab19905 | Within residues 100-200 |
| 5- | Mouse | Genway/20-783- | Methylated-cytosine |
| methylcytidine | monoclonal | 71663 | DNA/RNA |
| HDAC1 | Rabbit polyclonal | Abcam/ab19845 | residues 450 |
| | | | to the C-terminus |
| HDAC2 | Rabbit polyclonal | Abcam/ab32117 | Residues within C- |
| | | | terminal end |
| 5- | Mouse | Aviva Systems | Methylated -cytosine DNA |
| methylcytosine | monoclonal | Biology AMM99021 | |
| Pan methyl | Rabbit | Cell signaling/4473 | Methylated H3 |
| Histone H3 | monoclonal | | |
| TNF alpha | Mouse | Abcam/ab1793 | Full length native protein |
| | monoclonal | | |
| LIF | Mouse | Abcam/ab34427 | Recombinant full length |
| | monoclonal | | protein (human) |
| IL8 | Rabbit polyclonal | Abcam/ab7747 | Recombinant full length |
| | | | protein (human) |
| IL17Ra | Mouse | Abcam/ab92608 | Extracellular domain of |
| | monoclonal | | Human IL17A Receptor |
| IL10Ra | Mouse | Abcam/ab10777 | Recombinant full length |
| | monoclonal | | protein (human) |
| Complement C5 | Rabbit polyclonal | Abcam/ab66850 | Synthetic peptide conjugated |
| | | | to KLH derived from within |
| | | | residues 650-750 of Human |
| | | | C5 |
| H-Ferritin | Rabbit polyclonal | Abcam/ab65080 | Synthetic peptide conjugated |
| | | | to KLH derived from within |
| | | | residues 1-100 of H- |
| | | | Ferritin |
| L-Ferritin | Rabbit polyclonal | Abcam/ab69090 | Synthetic peptide conjugated |
| | | | to KLH derived from within |
| | | | residues 150 to the C- |
| | | | terminus of Human L- |
| | | | Ferritin |
| TfR | Mouse | Abcam/ab1086 | KG1 acute myelogenous |
| | monoclonal | | leukaemia cell line |
| GAPDH | Mouse | Abcam/ab8245 | Monomer (36 kDa) and also |
| | monoclonal | | the dimer forms of GAPDH |
| ∞-ACT | Rabbit polyclonal | Abcam/ab9374 | ∞-ACT isolated from human |
| | | | serum |
| HSP27 | Mouse | Abcam/ab2790 | Partially purified HSP 27 |
| | monoclonal | | derived from MCF-7 cytosol |
| HSP90 | Mouse | Abcam/ab13492 | Amino acid residues 604- |
| | monoclonal | | 697 of the human Hsp90 |
| | | | sequence |
| COX1 | Mouse | Abcam/ab695 | COX-1 |
| | monoclonal | | |
| COX2 | Goat polyclonal | Abcam/ab23672 | COX-2 |

In various embodiments, the present invention provides methods for determining a state of AD in a human patient. Accordingly, exemplary methods can comprise the steps of: receiving a blood sample from a patient; separating leukocytes from the blood sample; binding a first antibody to at least one epigenetic marker and at least one of an inflammation marker and a cellular stress marker in the leukocytes; conjugating a second antibody comprising a label to the first antibody; determining an amount of the label; applying a multivariate discriminant analysis to the amount of the label to produce an output value; and determining the state of AD in the patient based on the output value.

These methods can further comprise the step of adding EDTA to the blood sample, in which the separating the leukocytes can be by gravity. However in non-coagulated blood, the separating the leukocytes can be by centrifuge. As can be appreciated by those skilled in the art, EDTA when added to a blood sample can be at least one of a preservative and an anticoagulant.

These exemplary methods can comprise the steps of binding a third antibody to a second epigenetic marker and at least one of an inflammation marker and a cellular stress marker in a second portion of the leukocytes; conjugating a fourth antibody comprising a second label to the third antibody; determining an amount of the second label; applying a multivariate discriminant analysis to the amount of the second label to produce an output value; and determining the state of AD in the patient based on the amount of the label and the amount of the second label. These exemplary embodiments can comprise the step of comparing the amount of the label to a reference.

In various embodiments, proteomic techniques using mass spectrometry may be used to identify and quantify a particular protein or peptide, such as an epigenetic marker, and at least one of an inflammation marker and a cellular stress marker, in a protein extract derived from a biological sample. In one embodiment, the epigenetic marker, at least one of an inflammation marker and a cellular stress marker may be identified by comparing the theoretical mass to the mass of the proteins or peptides acquired experimentally in the sample using mass spectrometry. To determine the mass of a protein, its amino acid sequence may be submitted to proteomic software programs that determine the mass of proteins, peptides, and amino acids. These masses can then be compared to data generated by mass spectrometry analysis. In one embodiment, the sequence of an unknown isolated protein may be obtained by sequencing the protein with conventional amino acid sequencing techniques such as Edman degredation. The proteomic software program may then perform a virtual enzymatic digestion of the protein, such as with the enzyme trypsin, which cleaves proteins at known amino acid sequences, to produce peptide fragments. The resulting peptide fragments when run on a liquid chromatography mass spectrometry (LC-MS) system may produce a specific peptide mass fingerprint (PMF) that specifically identifies the protein it is derived from. In one embodiment, the PMF of an unknown isolated protein may be determined without sequencing by application of the digested protein to the mass spectrometer to determine the mass of its constituent peptides followed by a comparison of the peptide masses to protein database entries.

Once the PMF is obtained for the epigenetic marker and at least one of an inflammation marker and a cellular stress marker, the quantification of the markers from an actual biological sample may be determined. For example, a protein fraction from cell lysate samples may be digested with proteolytic enzymes that cleave proteins at specific locations. The resulting digested fragments may be introduced into a mass spectrometer by techniques such as matrix-assisted laser desorption and ionization (MALDI) or electrospray ionization (ESI-MS). These ionization techniques produce charged species which masses can be filleted and analyzed by mass analyzers, such as time of flights (TOFs), quadrupole, or ion trap, may determine the mass of the peptides. The data acquired by the mass spectrometer in combination with proteomic data analysis software programs can quantify marker levels in the samples when used with techniques such as added internal standards or spectral counting.

In various embodiments, antibodies may be used as a probe to identify particular molecules in cells, tissues, and biological fluids such as blood using immunofluorescence microscopy. A primary antibody that binds to a specific antigen, such as an epigenetic marker, and at least one of an inflammation marker and a cellular stress marker may be labeled directly by covalently binding a dye, such as a fluorescent molecule, to the primary antibody. More commonly, the binding of the primary antibody to the antigen may be detected by a secondary antibody labeled with a fluorescent molecule whose antigen is any other antibody. The labeled secondary antibody may be called a fluorescent anti-immunoglobulin. The fluorescent molecule may be excited by light at a particular wavelength, such as blue or green, resulting in the emission of light of a different wavelength for detection. The fluorescent molecule may comprise any number of conventional fluorescent molecules, such as green fluorescent protein from the jellyfish Aequorea Victoria. In an alternative embodiment to fluorescence, immunohistochemistry may be used in which the primary or secondary antibody is chemically coupled to an enzyme, such as horseradish peroxidase or alkaline phosphatase, which converts a colorless substrate into a colored reaction product in situ. The colored product identifying the epigenetic marker, the inflammation marker, and/or the cellular stress marker may be observed or quantified, such as by spectrometry methods.

In various embodiments, immunoblotting, also called Western blotting, may be used to identify the presence and quantity of an epigenetic marker, and at least one of an inflammation marker and a cellular stress marker in a cell lysate. A sample of cells, such as leukocytes, may be solubilized in a detergent to produce free solubilized proteins. The proteins may then be applied to a gel for gel electrophoresis to separate the proteins according to size. The proteins in the gel may be applied to a substrate such as a nitrocellulose membrane. The substrate may be treated with antibodies in which the antibodies bind their specific antigen on the membrane. The marker may then be viewed and quantified, such as by using a plate reader.

Similarly to the Western Blot, and in accordance with various embodiments, a protein dot blot methodology applies a protein fraction isolated from a cell lysate to a membrane, such as nitrocellulose, in a particular location or “spot.” However, the proteins are not first separated by gel electrophoresis. The protein spot may be treated with a labeled primary or secondary antibody to hybridize the antibody to the antigen, such as an epigenetic marker, an inflammation marker, or a cellular stress marker. Upon development of the fluorescent molecule or colored product identifying the marker, a quantitative measurement can be made of the spots using a spectrometer such as a plate reader.

In addition, according to various embodiments, an enzyme-linked immunosorbent assay (ELISA) may be used to detect an antigen, such as an epigenetic marker, and at least one of an inflammation marker and a cellular stress marker, using an antibody. To detect the antigen, the sample to be tested, such as a protein fraction from leukocytes, may be coated onto the surface of plastic wells. Labeled antibody, such as a primary or secondary antibody, may be added to the wells under conditions where nonspecific binding is prevented (called “blocking”), such that only binding to the antigen allows the antibody to be retained in the well after washing. The bound antibody may be detected by an enzyme-dependent color change or fluorescent reaction that may be observed and quantified by a spectrometer such as a multiwell plate reader.

Also, in accordance with various embodiments, a high throughput method of quantifying the amount of an epigenetic marker, an inflammation marker, and a in a biological sample, such as leukocytes isolated from a patient's blood, may comprise flow cytometry, such as fluorescence-activated cell sorting (FACS). Flow cytometry may be used to count the number of immunoreactive cells present in a sample by suspending the cells treated with labeled antibody in a stream of fluid, such as cell culture medium or buffer, and passing the cells by a fluorescence measuring system. The fluorescent properties of each cell may be determined to provide a graph, such as a histogram, indicating the various fluorescence intensities of all the cells in the sample. In one embodiment, threshold values may be applied to determine the presence of a disease state based on the percentage of cells that are immunoreactive in the sample.

Still further and in accordance to various embodiments, an epigenetic marker, and at least one of an inflammation marker and a cellular stress marker may be identified in a sample of cells, tissue, or a biological sample by visualization of labeled antibody bound to the marker using immunofluorescence microscopy. The sample may be applied to a microscope slide where a primary antibody is applied, such as the antibody diluted in a buffer in which the slide is submerged. Excess primary antibody may be washed away and a labeled secondary antibody may be applied to the slide. The slide may be viewed under a microscope, such as a fluorescence microscope or a confocal fluorescent microscope, configured to emit specific wavelengths of light onto the slide to produce fluorescence. In some embodiments, the intensity of fluorescence may be measured by a detector on the microscope to quantify the intensity of the fluorescence compared to a control sample.

According to various embodiments, methods can include quantifying an amount of an epigenetic marker, and at least one of an inflammation marker and a cellular stress marker in a sample. For example, using a dot blot assay for quantitative or qualitative examination of RNA and DNA immobilized on hybridization membranes is described herein. A quantitative dot blot for methylene blue stained nucleic acid may include blotting spots DNA or RNA in amounts of 1 μg, 0.8 μg, 0.6 μg, 0.4 μg, 0.2 μg, and 0.1 μg onto a hybridization membrane, such as for example nitrocellulose, as a calibration curve. The hybridization membrane is spotted with various dilutions of DNA or RNA extracted from blood leukocytes, followed by incubation of the membrane with methylene blue, followed by wash steps to remove excess stain, to detect total DNA or RNA. Signals are read by standard densitometry. Quantitation of the methylene blue calibration curve generates reference values for the quantitation of the DNA or RNA extracted from the leukocytes.

Various embodiments provide systems and apparatus that are useful for determining a state of AD in a patient. Accordingly, systems and/or apparatus can comprise a substrate comprising a top surface and a bottom surface; at least one detail on the top surface of the substrate; at least one antibody operative to bind to at least one epigenetic marker and at least one of an inflammation marker and a cellular stress marker in a sample comprising a leukocyte, the at least one antibody located in the at least one detail; and a reference value comprising a known amount of the at least one epigenetic marker and at least one of an inflammation marker and a cellular stress marker. These systems and/or apparatus can further comprise a second detail on the top surface of the substrate; a second antibody operative to bind a second epigenetic marker and a second at least one of an inflammation marker and a cellular stress marker in the sample comprising leukocyte, the second antibody can be located in the second detail; and a second reference sample comprising a known amount of the second epigenetic marker and a second at least one of an inflammation marker and a cellular stress marker. In one embodiment, the at least one detail is a spot and the at least one antibody is bound to the top surface of the substrate. In one embodiment, the at least one detail is a well and the at least one antibody is located in the well. In various embodiments, the sample comprises peripheral blood from a patient. In various embodiments, the reference value can be located in a reference detail located on the surface of the substrate and proximate to the at least one detail. These systems and/or apparatus can further comprise a label operable to identify the at least one epigenetic marker and the inflammation marker and/or the cellular stress marker. Still further, these exemplary systems and/or apparatus can further comprise a reader operable to measure an amount of the label. The systems and/or apparatus can comprise a cover sealing at least a portion of the top surface of the substrate.

In various embodiments, a system for determining a state of Alzheimer's disease, can comprise a substrate comprising a top surface and a bottom surface; at least one first detail and at least one second detail on the top surface of the substrate; at least one first label operative to bind at least one epigenetic marker in a sample comprising leukocyte, the at least one first label located in the at least one first detail; at least one second label operative to bind at least one of an inflammation marker and a cell stress marker in a sample comprising leukocyte, the at least one label located in the at least one second detail; a first reference value comprising a known amount of the at least one epigenetic marker; and a second reference value comprising a known amount of the at least one of the inflammation marker and the cell stress marker.

In various embodiments, the system can further comprise an apparatus for: quantifying signals detected from the at least first label and the at least one second label; calculating an amount of the at least one epigenetic marker and the at least one of the inflammation marker and the cell stress marker from the signals; performing a multivariate statistical analysis based on the amount of the at least one epigenetic marker and the at least one of the inflammation marker and the cell stress marker to produce a first output value a second output value; comparing the first output value and the second output value to the first reference value and the second reference value; calculating a risk score based on the comparison of the output value to the first reference value and the second reference value; and determining the patient's relative risk of developing Alzheimer's disease.

In various embodiments, the apparatus can further comprise a detector coupled to the substrate and adapted to receive the signals wherein the detector produces data representing the signals and transmit the data; a processor coupled to the detector and adapted to quantify the data representing the signals from the detector, and adapted to perform the multivariate statistical analysis, compare the output value to the first reference value and the second reference value, and calculate the risk score; and an output display coupled to the processor and configured to report the risk score. In one embodiment, the first reference value is located in a first reference detail on the top surface of the substrate and located proximate to the at least one first detail. In one embodiment, the second reference value is located in a second reference detail on the top surface of the substrate and located proximate to the at least one second detail.

Various embodiments include systems and/or apparatus that comprise a matrix that can detect a plurality of different epigenetic markers and at least one of a plurality of different inflammation markers and a plurality of different cellular stress markers from a plurality of sample portions. In one embodiment, the systems and/or apparatus cam further comprise a reference value for each of the plurality of different epigenetic markers and at least one of the plurality of different inflammation markers and the plurality of different cellular stress markers. In one embodiment, the reference value can be located proximate to the action region of the matrix. One embodiment includes a calibration curve proximate to each location to detect the plurality of different epigenetic markers and at least one of the plurality of different inflammation markers and the plurality of different cellular stress markers. Various embodiments described herein can be adopted for individual home use or in a hospital room or in a doctor's office.

In various embodiments, a kit can comprise an antibody to 5-methylcytosine, a peptide involved in DNA methylation, or a peptide involved in histone acetylation, a method to detect binding of the antibody directly (e.g., using a primary antibody that is conjugated to a fluorophor, enzyme, or coloring agent) or indirectly (e.g., secondary antibody conjugated to a fluorophor, enzyme, or coloring agent), and at one reference value corresponding to each of thresholds for various diagnoses of AD.

In various embodiments, a kit can comprise a stain or label which can comprise any moiety that can conjugate to an antibody that binds to an epigenetic marker, and at least one of an inflammation marker and a cellular stress marker. Still further, in one embodiment of the kit, the stain or the label can comprise an antibody that binds to an antibody that binds to an epigenetic marker, and at least one of an inflammation marker and a cellular stress marker. Furthermore, a kit can comprise the material to produce a calibration curve for a stain or label, however, the kit may comprise a premade standard calibration which can be used as a reference value. The kit can include various buffers and other reagents as described herein. Moreover, a kit can comprise an apparatus or systems described herein. Finally, kits can be designed to be especially useful for an individual's home use or a hospital use or use in a doctor's office.

The following are non-limiting examples of various embodiments of the present invention. It should be noted that any combination of materials discussed in these non-limiting examples may be included in a kit, in accordance with various embodiments.

### Example 1

In accordance with one embodiment, a quantitative real time reverse-transcription polymerase chain reaction (qRT-PCR) method for determining gene expression profiles of peripheral blood leukocytes is described. qRT-PCR is performed using the TaqMan® Gene Expression Assays (Applied Biosystems, Foster City, Calif., USA) and a GUSB Endogenous control assay. Each TaqMan® Gene Expression Assay is pre-formulated consisting of 2 unlabeled PCR primers at a final concentration of 900 nM and 1 FAM™ dye-labeled TaqMan® MGB of 250 nM final concentration. The following is a listing in Table 2 of primers, which are identified by Assay ID numbers and are available from Invitrogen (Carlsbad, Calif.), maybe useful for various Examples, described herein.

TABLE 2
Commercial Primers
TaqMan Gene Expression Assay List

| Gene Symbol (Name) | Assay ID |
|---|---|
| IL10RA (Interleukin 10 receptor, alpha) | Hs00387001_m1 |
| TNF (Tumor necrosis factor) | Hs99999043_m1 |
| LIF (Leukemia inhibitory factor) | Hs00171455_m1 |
| IL8 (Interleukin 8) | Hs01567912_g1 |
| IL17RA (Interleukin 17 receptor A) | Hs01064648_m1 |
| SERPING1 (C1 inhibitor) | Hs00934329_m1 |
| C5 (Complement component 5) | Hs01004343_m1 |
| DNMT1 (DNA methyltransferase 1) | Hs00945899_m1 |
| DNMT3A (DNA methyltransferase 3A) | Hs01027166_m1 |
| MBD2 (Methyl-CpG binding domain protein 2) | Hs00969372_m1 |
| HDAC1 (Histone deacetylase 1) | Hs00606262_g1 |
| HDAC6 (Histone deacetylase 6) | Hs00195869_m1 |
| SIRT1 (Sirtuin 1) | Hs01009006_m1 |
| SERPINA3 (alpha1-ACT) | Hs00153674_m1 |
| GAPDH (glyceraldehyde-3-phosphate dehydrogenase) | Hs02758991_g1 |
| HSPB1 (Heat shock protein 27) | Hs00356629_g1 |
| HSP90AB1 (Heat shock protein 90) | Hs00607336_gH |
| FTL (Ferritin L) | Hs00830226_gH |
| CRYAB (crystallin) | Hs00157107_m1 |
| PTGS1(COX1) | Hs00168776_m1 |
| PTGS2(COX2) | Hs00153133_m1 |
| TFRC (Transferrin R) | Hs00174609_m1 |
| GUSB (beta-glucuronidase) | Hs99999908_m1 |
| FTH1 (Ferritin H) | Hs01000478_g1 |

For each sample, 3.0 μg total RNA is reverse transcribed using the High Capacity cDNA Reverse Transcription Kit (Applied Biosystems). 2 μl of a 1:5 dilution for cDNA was combined with TaqMan® Universal PCR Master Mix No AmpErase UNG (Applied Biosystems) and the TaqMan® Gene Expression Assay in a 10 μl reaction set up by the CAS-1200 liquid handling system. The qRT-PCR reverse transcriptase amplifications were run on an ABI PRISM 7900 HT Sequence Detection System (Applied Biosystems). Universal thermal cycling conditions were as follows: 10 minutes at 95° C., 40 cycles of denaturation at 95° C. for 15 seconds, and annealing and extension at 60° C. for 1 minute. Amplifications efficiencies were close to 100% for all assays according to analyses of a number of different dilutions of the cDNA.

### Example 2

In accordance with one embodiment, a method of microarray analysis of gene expression in peripheral blood leukocytes is described. Patients that give informed consent are administered a battery of clinical dementia tests to determine AD subjects diagnosed with probable or possible AD on the basis of NINCDS (25) and DSM IV criteria for AD. Examination by a neurologist is performed to confirm diagnosis and to measure disease severity. Disease severity is assessed using the Mini-Mental Status Examination (MMSE, 11), the Clinical Dementia Rating scale (CDR; 17), and the Blessed Dementia Rating Scale (BDRS; 3). Control subjects included in the study scored above 27 on the MMSE, while AD cases scored below 22. The mean CDR of AD cases range between 1.2-1.5. Since the AD cases are not autopsy confirmed, the assignment of each case to a specified disease category relies on the accuracy of the clinical diagnosis. Thus, the clinical diagnosis is considered under the conventional nomenclature as “probable Alzheimer's disease”. Any subject with a history of bleeding diathesis or coagulopathy is excluded. Blood samples from both AD and control patients are drawn by a phlebotomist and stored at 4° C. until processed for RNA isolation (less than 8 hours).

Leukocyte RNA from each of these samples is extracted, yielding 4-6 μg RNA from 2.5 mL of blood. RNA quality and abundance was confirmed by a spectroscopic absorbance ratio with wavelengths of 260 nM:280 nM and by visualization with gel electrophoresis. RNA was hybridized to cDNA arrays and analyzed at different times.

The cDNA clones selected are those that are relevant in the field of AD research. Clones used to construct the arrays emphasized, but are not limited to, those that test the specific hypotheses that transcripts related to cellular stress, inflammation, and epigenetic mechanisms are be affected in leukocytes from AD cases. The dbEST database of the National Center for Biotechnology Information is searched for relevant 3′ cDNA clones.

1 μg of each linearized cDNA is denatured in 0.2 N NaOH/0.2 mM EDTA at 37° C. for 30 minutes. The sample volume is neutralized with 0.3 M NaOAc, pH 4.5. The cDNAs are immediately printed on a nylon membrane (Micron Separations) using a 96 pin replicator (Nalge Nunc) with each cDNA spotted four times.

PolyA-RNA is extracted from leukocytes using an mRNA isolation kit for blood (Roche). Erythrocytes are selectively lysed and leukocytes are collected by centrifugation. The leukocytes are then lysed and the total nucleic acids were collected by non-specific adsorption to magnetic glass beads and magnetic separation. Following a series of washes and elution of the nucleic acids from the magnetic glass beads, the mRNA is captured by the use of biotin-labeled oligo(dT) and streptavidin-coated magnetic particles. After removal of other nucleic acids (DNA, rRNA, tRNA) by washing, mRNA samples are collected and stored at −80° C. until later use. mRNA is amplified by the method described in Eberwine et al., Proc. Nat. Acad. Sci. 89, 3010-14 (1992) and radioactively labeled with ³²P CTP.

Before use, array membranes are prehybridized at 42° C. in hybridization solution (50% formamide/5×SSPE/5×Denhardt's solution/0.1% SDS/10% dextran sulfate/50 μg/mL denatured salmon sperm DNA/100˜g/ml tRNA) for 3 hours before adding the RNA probes. After overnight incubation at 42° C., blots are washed in 2×SSC/0.1% SDS at 55° C. for 1 hour, 2×SSC/0.1% SDS/10 μg/mL RNase A at 37° C. for 1 hour, and 2×SSC/0.1% SDS at 37° C. for 1 hour. Membranes are then exposed to a storage phosphor screen.

Hybridization intensity of each dot is detected by laser densitometric scanning (Phosphoimager, Molecular Dynamics). Values (counts) for each spot obtained by phosphoimager analysis are corrected using local background. The amount of cDNA deposited on each spot in the array is quantified by stripping and reprobing the membrane with an oligonucleotide specific for the T7 promoter present in all vectors. These data provided a correction for potential spot-to-spot differences in deposition of cDNA on the membrane. To ensure accurate comparisons across arrays, signals are normalized using the average of all markers (cDNAs) in an array for each RNA sample.

### Example 3

In accordance with one embodiment, a method of analyzing the standardized data is described. The standardized data are analyzed by two univariate tests and one multivariate test. The univariate tests are the t-test and the N test. The latter is essentially a non-parametric test for multiple testing inference. (Technical Report04/01 at http://www.urmc.rochester.edu/smd/biostat/people/techreports.html).

Multivariate statistical testing relies on canonical discriminant analysis. This analysis determines the variables (messages) that best distinguish groups and assigns weights to each variable. The first canonical variable provides the best distinction between groups. The second canonical variable operates on the residual variance that remains unaccounted for by canonical variable 1. Additional iterations are possible with diminishing effect. Valid results from multivariate canonical analyses requires that the number of variables analyzed (RNA messages in this case) be less than the number of cases (subjects) used. These two sets of transcripts were those related to either cellular stress (including, but not limited to, oxidative stress) or inflammation system. Two other subsets were formed, one composed of transcripts that approached significance in the t-test and one composed of transcripts chosen at random as a control for spurious results from the analytical methods used. These transcript sets are then utilized to analyze the data from three independent samples of early AD and control subjects.

A method of canonical discriminant analysis is used. This method is related to principal components analysis and is based on analysis of a correlation matrix that represents all the selected transcripts and all the cases. This analysis uses the canonical analysis program in the SAS/STAT® Software package. The analysis is instructed that there are two (or more) groups. It determines weights for the experimenter-selected transcripts and then assesses the ability of these weighted transcripts to distinguish groups. The weight determined for each transcript, and the expression level of that transcript are combined for each transcript for each person. The expression levels modified by weights for each transcript are combined to calculate an overall “risk score” for each person. This analysis also provides data on the weight assigned to each transcript in making the discrimination between AD and control. Follow-up clinical evaluations of the patient subjects are used to evaluate the leukocyte gene expression data as early indicators of future conversion to an AD disease state.

In the foregoing specification, the methods, apparatus, systems, and kits for detecting, diagnosing, predicting disease onset, and/or monitoring the progression of Alzheimer's disease has been described with reference to specific embodiments. Various modifications and changes may be made, however, without departing from the scope of the method, apparatus, system, and kits to detect, diagnose, predict the time for disease onset, and monitor the progression of AD as may be set forth in the claims. The specification and figures are illustrative, rather than restrictive, and modifications are intended to be included within the scope of the method and system to detect, diagnose, predict the time for disease onset, and monitor the progression of AD. Accordingly, the scope of the method and system to detect, diagnose, predict the time for disease onset, and monitor the progression of AD should be determined by the claims and their legal equivalents rather than by merely the examples described.

Benefits, other advantages and solutions to problems have been described with regard to particular embodiments; however, any benefit, advantage, solution to problem or any element that may cause any particular benefit, advantage or solution to occur or to become more pronounced are not to be construed as critical, required or essential features or components of any or all the claims in any issuing patent.

The terms “comprise”, “comprises”, “comprising”, “having”, “including”, “includes” and the like refer to a non-exclusive inclusion, such that a process, method, system, article, composition or apparatus that comprises a list of elements does not include only those elements recited, but may also include other elements not expressly listed or inherent to such process, method, system, article, composition or apparatus. Other combinations and/or modifications of the structures, arrangements, applications, proportions, elements, materials or components used in the practice of the method and system to detect, diagnose, predict the time for disease onset, and monitor the progression of AD, in addition to those not specifically recited, may be varied or otherwise particularly adapted to specific environments, manufacturing specifications, design parameters or other operating requirements without departing from the general principles of the same.

All literature and similar materials cited in this application, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

1. A method for determining a state of Alzheimer's disease in a patient, the method comprising:
obtaining a biological sample comprising at least one blood component from the patient;
determining an amount of expression for at least one epigenetic marker and at least one of an inflammation marker and a cellular stress marker in the biological sample;
applying a multivariate statistical analysis to the amount of expression to produce an output value;
comparing the output value to a reference value; and
determining a state of Alzheimer's disease in the patient.

2. The method according to claim 1, wherein comparing the output value to the reference value results in a risk score representing the patient's relative risk of developing Alzheimer's disease.

3. The method according to claim 2, wherein the risk score distinguishes the state of Alzheimer's disease from a non-disease state in the patient from another neurological disease state in the patient.

4. The method according to claim 1, further comprising applying a label to the sample to identify the at least one epigenetic marker and the at least one of an inflammation marker and a cellular stress marker

5. The method according to claim 4, further comprising placing the biological sample comprising at least one blood component onto a substrate.

6. The method according to claim 4, wherein determining an amount of the at least one epigenetic marker and the at least one of the inflammation marker and the cellular stress marker comprises quantifying an intensity of the label.

7. The method according to claim 1, wherein the at least one epigenetic marker and the at least one of an inflammation marker and the cellular stress marker are at least one epigenetic marker and at least one the inflammation marker and at least one cell stress marker.

8. The method according to claim 1, further comprising preparing a treatment plan for the patient based on the state of Alzheimer's disease.

9. The method according to claim 8, further comprising treating the patient with a therapeutic substance.

10. The method according to claim 1, wherein the at least one epigenetic marker is at least one of a DNA methylation marker, a histone modification marker, a methylated DNA binding protein marker, and a deacetylase marker.

11. The method according to claim 1, wherein the at least one inflammation marker is at least one of a complement marker, a cytokine receptor marker, and a cytokine marker.

12. The method according to claim 1, wherein the at least one cell stress marker is at least one of an iron-binding protein marker, a cellular metabolism marker, a protein chaperone marker, a cyclooxygenase marker, and a protease inhibitor marker.

13. The method according to claim 1, further comprising:
placing a second biological sample comprising the at least one blood component onto the substrate;
determining a second amount of expression of the at least one epigenetic marker and the at least one of an inflammation marker and a cellular stress marker;
comparing the second amount of expression of the at least one epigenetic marker and the at least one of the inflammation marker and the cellular stress marker to at least one second reference value;
applying a multivariate statistical analysis to the second amount of the at least one second reference value compared to the second amount of the at least one epigenetic marker and the at least one of the inflammation marker and the cellular stress marker to produce a second output value; and
determining a dosage of a therapeutic substance.

14. The method according to claim 13, further comprising evaluating the efficacy of the therapeutic substance.

15. The method according to claim 14, wherein evaluating the efficacy of the therapeutic substance further comprises comparing the amount of the at least one epigenetic marker and the at least one of the inflammation marker and cellular stress marker over a period of time.

16. The method according to claim 13, further comprising determining an updated state of Alzheimer's disease in the patient.

17. The method according to claim 16, further comprising developing a treatment plan based on the updated state of Alzheimer's disease in the patient.

18. A method for determining the probability of developing Alzheimer's disease in a patient before the onset of symptoms, the method comprising:
obtaining a biological sample comprising at least one blood component from the patient;
determining an expression level of at least one epigenetic marker and at least one of an inflammation marker and a cellular stress marker;
applying a multivariate statistical analysis to the expression level of the at least one epigenetic marker and the at least one of the inflammation marker and the cellular stress marker to produce an output value;
comparing the output value to a reference value; wherein comparing the output value to the reference value results in a risk score; and
determining the patient's risk of developing Alzheimer's disease based on the risk score.

19. The method according to claim 18, wherein the at least one epigenetic marker is at least one of a DNA methylation marker, a histone modification marker, a methylated DNA binding protein marker, and a deacetylase marker.

20. The method according to claim 18, wherein the at least one inflammation marker is at least one of a complement marker, a cytokine receptor marker, and a cytokine marker.

21. The method according to claim 18, wherein the at least one cell stress marker is at least one of an iron-binding protein marker, a cellular metabolism marker, a protein chaperone marker, a cyclooxygenase marker, and a protease inhibitor marker.

22. The method according to claim 18, wherein the at least one epigenetic marker and the at least one of an inflammation marker and the cell stress marker are at least one epigenetic marker and at least one the inflammation marker and at least one cell stress marker.

23. The method according to claim 18, further comprising determining the patient's risk of developing Parkinson's disease based on the risk score.

24. A system for determining a state of Alzheimer's disease in a sample, the system comprising:
a substrate comprising a top surface and a bottom surface;
at least one first detail and at least one second detail on the top surface of the substrate;
at least one first label operative to bind to at least one epigenetic marker in a sample comprising leukocyte, the at least one first label located in the at least one first detail;
at least one second label operative to bind to at least one of an inflammation marker and a cellular stress marker in a sample comprising leukocyte, the at least one label located in the at least one second detail;
a first reference value comprising a known amount of the at least one epigenetic marker; and
a second reference value comprising a known amount of the at least one of the inflammation marker and the cellular stress marker.

25. A system according to claim 24, the system further comprising an apparatus for:
quantifying signals detected from the at least one first label and the at least one second label;
calculating an amount of the at least one epigenetic marker and the at least one of the inflammation marker and the cellular stress marker from the signals;
performing a multivariate statistical analysis based on the amount of the at least one epigenetic marker and the at least one of the inflammation marker and the cellular stress marker to produce a first output value and a second output value;
comparing the first output value and the second output value to the first reference value and the second reference value;
calculating a risk score based on the comparison of the first output value and the second output value to the first reference value and the second reference value; and
determining a patient's relative risk of developing Alzheimer's disease.

26. The system according to claim 25, wherein the apparatus comprises:
a detector coupled to the substrate and adapted to receive the signals wherein the detector produces data representing the signals and transmit the data;
a processor coupled to the detector and adapted to quantify the data representing the signals from the detector and adapted to perform the multivariate statistical analysis, compare the output value to the first reference value and the second reference value, and calculate the risk score; and
an output display coupled to the processor and configured to report the risk score.

27. The system according to claim 24, wherein the first reference value is located in a first reference detail on the top surface of the substrate and located proximate to the at least one first detail.

28. The system according to claim 24, wherein the second reference value is located in a second reference detail on the top surface of the substrate and located proximate to the at least one second detail.
